(19) United States
(12) Patent Application Publication
(10) Pub. No.: US 2013/0157974 A1
(43) Pub. Date: Jun. 20, 2013

(54) Preparation Comprising Iron(III) Complex Compounds And Redox-Active Substance(s)

(71) Applicant: VIFOR (INTERNATIONAL) AG, St. Gallen (CH)

(72) Inventors: Priska Ziegler, Beckenried (CH); Peter Geisser, St. Gallen (CH)

(73) Assignee: VIFOR (INTERNATIONAL) AG, St. Gallen (CH)

(21) Appl. No.: 13/735,228

(22) Filed: Jan. 7, 2013

Related U.S. Application Data
(62) Division of application No. 12/092,721, filed on May 6, 2008.

(30) Foreign Application Priority Data
Nov. 24, 2005 (EP) 05111229.0

(51) Int. Cl. A61K 31/715; A61K 31/375; A61K 45/06

(52) U.S. Cl. CPC A61K 31/715; A61K 45/06; A61K 31/375
USPC 514/54

(57) ABSTRACT

A preparation is disclosed that comprises one or more iron(III) complex compounds which have a redox potential at pH 7 of from −324 mV to −750 mV relative to a normal hydrogen electrode (NHE), and one or more redox-active substances, wherein the carbohydrates are selected from the group consisting of dextrans and hydrogenated dextrans, dextrins, oxidised or hydrogenated dextrins, as well as pullulan, oligomers thereof and/or hydrogenated pullulans, and wherein the redox-active substance(s) is/are selected from the group consisting of ascorbic acid; vitamin E; cysteine; physiologically acceptable phenols/polyphenols selected from the group consisting of quercetin, rutin, flavones, flavonoids, hydroquinones; and glutathione, and in particular is ascorbic acid.

The present invention relates to a preparation comprising iron(III) complex compounds that have a specific redox potential, in particular with carbohydrates or derivatives thereof, in particular with dextrins or oxidation products of dextrins, and one or more redox-active substance(s), in particular ascorbic acid, as well as optionally further vitamins, trace elements, minerals, nutrients and/or cofactors, and also to the use thereof as a medicament for the treatment of iron deficiency states and further diseases, and to the use of the iron(III) complex compounds in the preparation of a medicament for the treatment of iron deficiency states and further diseases, wherein the medicament is administered simultaneously with or close in terms of time to redox-active substance(s).

Iron deficiency is the most common trace element deficiency worldwide. About two thousand million people worldwide suffer from iron deficiency or iron deficiency anaemia (E. M. DeMaeyer, “Preventing and controlling iron deficiency anaemia through primary health care”, World Health Organization, Geneva, 1989, ISBN 92 4 154249 7).

The use of iron(III) oxide as an active ingredient for the treatment of immune deficiency syndromes, in particular AIDS, is known from WO 95/35113.

Therapeutically usable iron injection preparations and processes for their preparation are known from DE 1467980.

Processes for the preparation of iron(III)-polymaltose complex compounds which are suitable for parenteral administration are known from U.S. Pat. No. 3,076,798.

The use of iron-carbohydrate complexes in the treatment or prophylaxis of iron deficiency states is known from WO 04/037865.

Iron complex compounds with hydrogenated dextrins for the treatment or prophylaxis of iron deficiency states are known from WO 03/087164.

Iron(III)-pullulan complex compounds and their use in the treatment or prophylaxis of iron deficiency states are known from WO 02/46241.

WO 99/48533 discloses iron-dextran compounds for the treatment of iron deficiency anaemia that comprise hydrogenated dextran having a specific molecular weight of approximately 1000 daltons.

WO 01/00204 discloses anti-anaemic compositions which comprise Fe(III) complexes of hydroxamates, hydroxy-pyridinones and siderophores (catecholamides) and vitamin C and/or E for protecting against oxidative stress and dysfunction of the endothelium. The redox potential of the iron(III) complexes that are used is not discussed.

U.S. Pat. No. 4,994,283 discloses compositions comprising iron(II)- or iron(III)-sugar complexes and ascorbate in the form of fruit juice, for the treatment of anaemias.

WO 2004/082693 discloses compositions comprising iron(II) and iron(III) complexes, for example, with carbohydrates such as dextran, dextrin/polymaltose and, in particular, sucrose, for the treatment of restless legs syndrome. Conventional adjuvants such as ascorbic acid can be added to the composition.

EP-A-0 134 936 discloses hydrotalcite-like complexes comprising iron(III) for the treatment of anaemias, for example in drink form. Further additives are, for example, glucose and preferably ascorbic acid and glutathione.

It is known that iron sulfate relatively frequently causes unpleasant dose-dependent secondary reactions, such as gastrointestinal disturbances or discolouration of the teeth. Iron from iron salt compounds is subject to the passive diffusion of free iron ions. The iron can enter the circulation and thus cause secondary reactions or iron poisoning. Accordingly, even the LD50 value in white mice, at 230 mg of iron/kg, is relatively low.

The use of iron-dextran is disclosed in Oski et al. “Effect of Iron Therapy on Behavior Performance in Nonanemic, Iron-Deficient Infants”, PEDIATRICS 1983; Volume 71; 877-880. The parenteral use of iron-dextran is disadvantageous because a dextran-induced anaphylactic shock can occur.

Conventional oral iron preparations, generally iron(II) salts, frequently cause severe gastrointestinal side-effects, which leads to poor patient compliance. Oral iron therapy can increase the lesions of the intestinal tissue by catalysing the formation of reactive oxygen species. Because free iron is a strong catalyst of the formation of reactive oxygen species, oral iron(II) therapy can even be harmful, in particular for patients with chronic inflammatory bowel disease. Oral iron(II) preparations are poorly absorbed and result in high faecal iron concentrations, and a significant proportion of the faecal iron is available for the catalytic activity. When iron comes into contact with intestinal mucosa, which may already be inflamed, it can increase the production of reactive oxygen species and thus increase tissue damage.

Iron(III)-polymaltose complex contains iron in non-ionic form, which is less toxic. When compounds of this type are administered, fewer side-effects occur and patient compliance is improved as compared with iron(II) sulfate (Jacobs, P., Wood, L., Bird, A. R., Hematol. 2000, 5:77-83).

Many different research results have shown that the amount of ascorbic acid present in food influences the absorption of iron, and that the addition of ascorbic acid to food greatly improves the bioavailability of the iron contained in the food (e.g. Björn-Rasmussen E. et al., Nutr. Metabol. 1974, 16, 94-100; Cook J. D. et al., Am. J. Clin. Nutr. 1977, 30, 235-241; Derman D. P. et al., Scand. J. Haematol. 1980, 25, 193; Gillooly C. et al., Scand. J. Haematol. 1982, 29, 18-24; Hallberg L., Ann. Rev. Nutr. 1981, 1, 123-147; Hallberg, L. et al., Am. J. Clin. Nutr. 1984, 39, 577; Morch, T. A. et al., Am. J. Clin. Nutr. 1982, 36, 219-223; Sayers M. H., Br. J. Haematol. 1973, 24, 209-218; Sayers M. H. et al., Br. J. Nutr. 1974, 31, 367-375; Sayers M. H. et al., Br. J. Haematol. 1972, 28, 483-495). It is often assumed that the reduction of the poorly soluble, trivalent iron to readily soluble divalent iron plays a deciding role.

In consideration of this, special iron preparations comprising ascorbic acid were developed, and these are well represented on the market today. These preparations are combinations of iron(II) salts, predominantly iron(II) sulfate, with ascorbic acid. The ascorbic acid in these preparations serves to prevent the oxidation of iron(II) to iron(III) in the preparation.

It is further known that iron(II) salts form a coloured complex with ascorbic acid, and that ascorbic acid forms a soluble chelate complex with iron(III) chloride at acidic pH, but not with iron(III) precipitates at alkaline pH. The soluble iron(III)-chelate complex is stable and contains iron in soluble form even if the solution is subsequently rendered alkaline (Conrad, M. E. et al., Gastroenterology 1968, 55, 35-45).

By means of Mössbauer and UV/VIS spectroscopy under oxygen-free conditions it has been possible to show that, in the pH range 6-7, ascorbic acid forms complexes not with Fe²⁺ but with Fe³⁺ (Hamed, M. Y. et al., Inorg. Chim. Acta 1988, 152, 227-231). Fe²⁺ does not form complexes with ascorbic acid, and precipitations are therefore observed in the alkaline range, in contrast to the system Fe³⁺ with ascorbic acid, which is present in the form of a Fe(III) complex solution at neutral and alkaline pH (Gorman, J. E. et al., J. of Food Science 1983, 48, 1217-1225). Fe³⁺ forms a red, water-soluble 1:1 complex with ascorbic acid at pH 6.5 (Hamed, M. Y. et al., Inorg. Chim. Acta 1988, 12, 227-231).

Iron(III) complexes can be divided into the following two groups:
those which can be reduced under physiological conditions (pH 7) with NADP(H) to iron(II)
those which cannot be reduced under those conditions.

The critical redox potential therefor is −324 mV. This is the redox potential of NAD(P)H/NADP⁺ at pH 7.

It is known that the redox potential for the reaction of ascorbic acid to dehydroascorbic acid at pH 7 is −66 mV (Borsook, H. et al., Proc. N.A.S. 1933, 875-878). This means that iron(III) complexes having a redox potential of <−66 mV at pH 7 cannot be reduced by ascorbic acid. Iron(III)-polymaltose complex has a redox potential of −332 mV at pH 7 (Crichton, R. R., Danielson, B. G., Geisser, P. Iron Therapy with special emphasis on intravenous administration, 2nd edition, UNI-MED Verlag AG, Bremen, 2005, p. 44, FIG. 6.4).

Throughout the present patent application, all redox potentials are always measured and quoted relative to a normal hydrogen electrode (NHE).

Older studies have already questioned the reduction of iron³⁺ to iron²⁺ under gastrointestinal conditions (Gorman, J. E. et al., J. of Food Science 1983, 48, 1217-1225). More recent studies show that a reduction occurs at a strongly acidic pH, but the formation of an iron(III)-ascorbic acid complex is the quicker reaction and can take place even at higher pH values (Dorey, C. et al., Iron Club Meeting 1988; Xu, J. et al., Inorg. Chem. 1990, 29, 4180-4184).

As early as 1968 it was shown that FeCl₂ and FeCl₃ exhibit far better absorption results in combination with ascorbic acid than without (Conrad, M. E. at al., Gastroenterologie 1968, 55, 35-45). In concrete terms, this study showed that Fe(III) from FeCl₃ is resorbed about equally as well as Fe(II) from FeCl₂, that ascorbic acid brings about an absorption-increasing effect for Fe(II) and Fe(III), that, of the preparations studied, the Fe(III)-ascorbic acid complex exhibits the best absorption, and that the corresponding preparation exhibits higher absorption than the other preparations studied, not only in anaemic rats but also in non-anaemic rats.

Derman (Derman, D. P. et al., Scand. J. Haematol. 1980, 25, 193-201) was able to show that the absorption of 3 mg of Fe as ferritin or iron(III) hydroxide from a maize porridge meal is improved by the addition of 100 mg of ascorbic acid from in each case 0.4% to 12.1 and 10.5%, respectively. The proportion of dialysable iron could be improved considerably by complex formation with ascorbic acid at pH 3.0 and 7.0.

It is further known that acid does not increase the absorption of iron(II) salts or haemoglobin iron but might increase the absorption of iron from iron(III) salts and from food (Conrad, M. E. et al., Gastroenterology 1968, 55, 35-45). Ionic iron(III) is not absorbed by the intestinal mucosa, and gastrointestinal secretions must therefore first reduce ionic iron(III) to ionic iron(II) or chelate it in order to dissolve the iron and increase its absorption.

A further study by Naito (Naito, Y. et al., Digestion 1995, 56, 472-478) has shown that iron(II) ions in combination with ascorbic acid cause local ulcerations in the gastro-intestinal tract, and that oxygen-radical-mediated lipid peroxidation plays a deciding role in the pathogenesis of gastric ulcerations caused by iron(II) in combination with ascorbic acid.

It was earlier assumed that high concentrations of ascorbic acid inhibit lipid peroxidation, presumably owing to direct antioxidation properties (Bucher, J. R., et al., Fund. Appl. Tox. 1983, 3, 222-226), but also by keeping iron solely in reduced form (Baughler, J. M. at al., J. Biol. Chem. 261, 10282-10289).

However, iron(II) reacts with oxygen to give iron(III) and free OH radicals. The formation of these radicals leads to intensive side-effects and is undesirable. A study by Fodor recently showed (Fodor, I. at al., Biochim. Biophys. Acta 961 (1988), 96-102) that the combination of iron(II) with ascorbic acid leads to significantly more ulcerations in the gastrointestinal tract than does iron(II) alone. This study has further shown that ascorbic acid in combination with iron(II) is a promoter of lipid peroxidation and not an inhibitor, as earlier assumed, because, of all the combinations studied, the combination of iron(II) with ascorbic acid induced the most intensive lipid peroxidation. Lipid peroxidation is in turn responsible for damage to biological membranes and hence for ulcerations.

Further studies have also consistently shown the combination of iron(II) and ascorbic acid to be toxic (Higson, F. K., et al., Free Rad. Res. Comms. 1988, 5, 107-115; Uchida, K. et al., Agric. Biol. Chem. 1989, 53, 3285-3292). The damage that occurs at cell level as a result of these toxic effects then causes side-effects and poor patient compliance.

Oxidative stress, in particular lipid peroxidation, is associated, for example, with an increased risk of. suffering from heart attack, cancer and atherosclerosis. The oxidative modification of low-density lipoprotein (LDL) is held responsible for atherogenesis (see references quoted in Tuomainen et al., Nutrition Research, Vol. 19, No. 8, pp. 1121-1132, 1999).

The inventors have therefore set themselves the object of finding readily tolerable iron(III) preparations in combination with one or more redox-active substance(s), which preparations are suitable for the treatment of iron deficiency states and ensure improved bioavailability of the iron without exhibiting the above-described disadvantageous effects of the known iron(II)-ascorbic acid combination preparations, such as the formation of ulcerations in the gastrointestinal tract and oxidative stress due to lipid peroxidation.

The object of the invention was, therefore, to provide a combination of iron(III) and one or more redox-active substance(s) in which the iron(III) is not reduced to iron(II) by the redox-active substance(s), in particular ascorbic acid, and accordingly does not cause oxidative stress. The optimum absorption possibility for iron by formation of iron(III) complexes with the redox-active substance(s) should, on the other hand, be utilised.

The object is achieved by the preparation according to the invention, which comprises iron(III) complex compounds having a specific redox potential, in particular iron(III) complex compounds with carbohydrates or derivatives thereof, and one or more redox-active substance(s); in particular ascorbic acid.

Iron(III) complex compounds with carbohydrates, in particular with polymaltose (maltodextrin), are particularly tolerable and have high patient compliance. No oxidative stress occurs during treatment with the iron(III) complexes.

Studies by the inventors have shown that iron(III)-polymaltose complex, which has a reduction potential at pH 7 of −332 mV, does not react with ascorbic acid to give dehydroascorbic acid at pH 3, 5.5 and 8 in buffered solution (buffer systems pH 3.0: 10⁻³ mol/l HCl; pH 5.5 and pH 8.0: 0.1 mol/l NH₄Cl/NH₃; see Geisser, P., Arzneim.—Forsch./Drug Res. 1990, 40 (II), 7, 754-760) with the exclusion of oxygen.

Although iron(III)-polymaltose complex compounds only result in a slow increase in the ferritin level, they are used more efficiently for haemoglobin synthesis (T.-P. Tuomainen et al., loc. cit., p. 1127).

According to the invention, an iron deficiency state is understood as being a state in which haemoglobin, iron and ferritin are reduced in the plasma and transferrin is increased, which results in reduced transferrin saturation.

The condition to be treated in accordance with the invention includes iron deficiency anaemia and iron deficiency without anaemia. The division can be made, for example, by the haemoglobin value and the value for transferrin saturation (%). Reference values for haemoglobin, determined by flow cytometry or by the photometric cyanohaemoglobin method, and reference values for iron, ferritin and transferrin are listed, for example, in the reference databank of Charité, Institut für Laboratoriumsmedizin und Pathobiochemie (http://www.charite.de/ilp/routine/parameter.html) and in Thomas, L. Labor und Diagnose, TH Book Verlagsgesellschaft, Frankfurt/Main 1998. In patients without iron deficiency, transferrin saturation is generally >16%. In patients without iron deficiency, the ferritin value is generally at least 30 μg/1 and the haemoglobin value is at least 130 g/l.

According to M. Wick, W. Pinggera, P. Lehmann, Eisenstoff-wechsel—Diagnostik and Therapien der Anämien, 4th extended edition, Springer Verlag Vienna 1998, all forms of iron deficiency can be detected clinico-chemically. A reduced ferritin concentration is generally accompanied, by way of compensation, by increased transferrin and low transferrin saturation.

The preparation according to the invention can further be used for improving immune defence and for improving brain power.

Improvement in immune defence within the scope of the invention means a significant improvement in the immune responses as shown, for example, by a significant improvement in the lymphocyte response to phytohaemagglutinin (PHA) using the MTT method, by an improvement in the nitroblue tetrazolium test (MBT) using neutrophils, by an improvement in the bactericidal capacity of neutrophils (PCA) measured by the turbidimetric process, by an improvement in monoclonal antibodies, for example CD3, CD4, CD8 and CD56, counted, for example, using a BD flow-cytometer with a simple staining method, and/or in the antibody response to measles, H. influenza and tetanus. In the last-mentioned cases, the use according to the invention takes place in particular by improving the neutrophil level, the antibody level and/or the lymphocyte function, determined, for example, by the lymphocyte reaction to phytohaemagglutinin.

An improvement in brain power within the scope of the invention includes in particular an improvement in cognitive functions and emotional behaviour and is expressed, for example, in an improvement in the short-term memory test (STM), in the long-term memory test (LTM), in the Raven's progressive matrices test, in the Welscher adult intelligence scale (WAIS) and/or in the emotional coefficient (Baron EQ-i, YV test; youth version).

The preparation according to the invention can also be used in the treatment of restless legs syndrome (RLS, also known as Ekbom's syndrome). This is a disease in which patients are unable to sit still or even stand still. Because of the irresistible urge to move, patients also suffer from pronounced insomnia. As soon as the patient moves, the symptoms disappear, but they return immediately when the movement stops. If patients are forced to lie down, involuntary leg movements are observed. For further explanations of this indication, reference is made to WO 2004/083693.

The preparation according to the invention is further suitable for the treatment of iron deficiency states in patients with chronic inflammatory bowel diseases, in particular Crohn's disease and Colitis ulcerosa.

The preparation according to the invention is also particularly suitable for treating or preventing iron deficiency states in pregnant women, in particular when it contains further pharmacologically active constituents in the form of vitamins, with the exception of ascorbic acid, minerals, trace elements, nutrients and/or trace elements, as described hereinbelow.

Iron(III) complex compounds which can be used in accordance with the invention are those having a redox potential at pH 7 of from −324 mV to −750 mV, preferably from −330 mV to −530 mV, particularly preferably from −332 mV to −475 mV. These conditions are fulfilled in particular by specific iron(III)-polymaltose complexes, iron(III)-dextrin complexes, iron(III)-dextran complexes and iron(III)-sucrose complexes as well as by the iron(III)-transferrin complex. Of these, iron(III)-polymaltose complexes having the mentioned redox potential are particularly preferred. However, other iron(III) complex compounds are also suitable, provided they have a redox potential within the mentioned range.

Iron(III) complex compounds which can be used in accordance with the invention are in particular those with carbohydrates. They preferably include those wherein carbohydrates are selected from the group consisting of dextrans and derivatives thereof, dextrins and derivatives thereof, and pullulan, oligomers and/or derivatives thereof. The mentioned derivatives include in particular the hydrogenated derivatives. Iron(III) complex compounds with dextrins or oxidation products thereof are particularly preferred. Examples of the preparation of the iron(III) complex compounds according to the invention will be found, for example, in the patent specifications DE 14679800, WO 04037865 A1, U.S. Pat. No. 3,076,798, WO 03/087164 and WO 02/46241 mentioned at the beginning, the totality of the disclosures of which, in particular in respect of the preparation processes, is to be incorporated herein. The term “dextrins”, which are preferably used in accordance with the invention, is a collective term for various lower and higher polymers of D-glucose units, which form when starch is incompletely hydrolysed. Dextrins can also be prepared by polymerisation of sugars (e.g. WO 02083739 A2, US 20030044513 A1, U.S. Pat. No. 3,766,165). The dextrins include the maltodextrins, or polymaltoses, which are prepared by enzymatic cleavage of, for example, corn or potato starch with alpha-amylase and are characterised by the degree of hydrolysis, which is expressed by the DE value (dextrose equivalent). Polymaltose can also be obtained according to the invention by acidic hydrolysis of starches, in particular. of dextrins. The preparation of the iron(III) complex compounds which can be used in accordance with the invention generally takes place by reaction of iron(II) or iron(III) salts, in particular iron(III) chloride, with the dextrins, in particular polymaltose, or oxidation products of the dextrins in aqueous alkaline solution (pH>7) and subsequent working up. Preparation in the weakly acidic pH range is also possible. However, alkaline pH values of, for example, >10 are preferred.

The pH value is preferably increased slowly or gradually, which can be effected, for example, by first adding a weak base, for example to a pH of approximately 3; further neutralisation can then be carried out using a stronger base. Suitable weak bases are, for example, alkali or alkaline earth carbonates, bicarbonates, such as sodium and potassium carbonate or bicarbonate, or ammonia. Strong bases are, for example, alkali or alkaline earth hydroxides, such as sodium, potassium, calcium or magnesium hydroxide.

The reaction can be furthered by heating. For example, temperatures of the order of magnitude of from 15° C. to the boiling temperature can be used. It is preferred to increase the temperature gradually. For example, heating can first be carried out to approximately from 15 to 70° C. and then the temperature can be gradually increased to boiling.

The reaction times are, for example, of the order of magnitude of from 15 minutes to several hours, e.g. from 20 minutes to 4 hours, for example from 25 to 70 minutes, e.g. from 30 to 60 minutes.

When the reaction has been carried out, the resulting solution can, for example, be cooled to room temperature and optionally diluted and optionally filtered. After cooling, the pH value can be adjusted to the neutral point or slightly below, for example to values of from 5 to 7, by addition of acid or base. As bases there can be used, for example, those mentioned above for the reaction. Acids include hydrochloric acid and sulfuric acid, for example. The resulting solutions are purified and can be used directly for the preparation of medicaments. However, it is also possible to isolate the iron(III) complexes from the solution, for example by precipitation with an alcohol, such as an alkanol, for example ethanol. Isolation can also be effected by spray drying. Purification can be carried out in a conventional manner, in particular in order to remove salts. This can be effected, for example, by reverse osmosis, it being possible for such a reverse osmosis to be carried out, for example, before the spray drying or before the direct use in medicaments.

The resulting iron(III) complexes have, for example, an iron content of from 10 to 40% wt./wt., in particular from 20 to 35% wt./wt. They are generally readily soluble in water. It is possible to prepare therefrom neutral aqueous solutions having an iron content of, for example, from 1% wt./vol. to 20% wt./vol. Such solutions can be sterilised by means of heat.

With regard to the preparation of iron(III)-polymaltose complex compounds, reference may also be made to U.S. Pat. No. 3,076,798.

In a preferred embodiment of the invention, an iron(III) hydroxide-polymaltose complex compound is used. This iron(III)-polymaltose complex compound preferably has a molecular weight in the range from 20,000 to 500,000, in a preferred embodiment from 30,000 to 80,000 daltons (determined by means of gel permeation chromatography, for example as described by Geisser et al. in Arzneim. Forsch./Drug Res. 42(11), 12, 1439-1452 (1992), Section 2.2.5.). A particularly preferred iron(III) hydroxide-polymaltose complex compound is Maltofer® from Vifor (International) AG, Switzerland, which is available commercially. In a further preferred embodiment, an iron(III) complex compound with an oxidation product of one or more maltodextrins is used. This is obtainable, for example, from an aqueous iron(III) salt solution and an aqueous solution of the product of the oxidation of one or more maltodextrins with an aqueous hypochlorite solution at a pH value in the alkaline range, wherein when one maltodextrin is used its dextrose equivalent is from 5 to 37 and when a mixture of a plurality of maltodextrins is used the dextrose equivalent of the mixture is from 5 to 37 and the dextrose equivalent of the individual maltodextrins in the mixture is from 2 to 40. The weight-average molecular weight Mw of the complexes so obtained is, for example, from 30 kDa to 500 kDa, preferably from 80 to 350 kDa, particularly preferably up to 300 kDa (determined by means of gel permeation chromatography, for example as described by Geisser et al. in Arzneim. Forsch./Drug Res. 42(11), 12, 1439-1452 (1992), Section 2.2.5.). Reference may be made in this connection to WO 2004037865 A1, for example, the totality of the disclosure of which is to be incorporated in the present application.

With regard to the preparation of iron complex compounds with hydrogenated dextrins, reference may be made to WO 03/087164.

With regard to the preparation of iron(III)-pullulan complex compounds, reference may be made to WO 02/46241.

As redox-active substance(s) there can be used in accordance with the invention ascorbic acid, vitamin E, cysteine, physiologically acceptable phenols/polyphenols and glutathione. Suitable physiologically acceptable phenols/polyphenols are, for example, quercetin, rutin, flavones, other flavonoids (e.g. campherols) and hydroquinones, in particular quercetins, as well as derivatives of the mentioned compounds. Ascorbic acid is particularly preferred. One or more of these redox-active substances can be used; particular preference is given to the combination of vitamin E with ascorbic acid and ascorbic acid alone.

In the preparation according to the invention, the iron(III) complex compound and the redox-active substance(s), in particular ascorbic acid, are preferably present in a weight ratio of from 1:0.05 to 1:20, preferably from 1:0.3 to 1:2, particularly preferably from 1:0.4 to 1:1.8, most preferably 1:1.5 (based on the iron(III) complex compound, not on iron(III)).

The preparation according to the invention can optionally comprise further pharmacologically active constituents which are selected from the group consisting of vitamins, with the exception of ascorbic acid, trace elements, minerals, nutrients and cofactors. The further pharmacologically active constituents are preferably the vitamins β-carotene, thiamine (vitamin B₁), riboflavin (vitamin B₂), pyridoxine (vitamin B₆), cyanocobalamin (vitamin B₁₂), cholecalciferol (vitamin D₃), α-tocopherol (vitamin E), biotin. (vitamin H), the cofactors pantothenic acid, nicotinamide, folic acid, the trace elements/minerals copper, manganese, zinc, calcium, phosphorus and/or magnesium, and the nutrients amino acids, oligopeptides, carbohydrates and fats, optionally in the form of physiologically acceptable salts. Suitable physiologically acceptable salts are any conventional physiologically acceptable salts, preferably salts of inorganic acids or bases, such as hydrochlorides, sulfates, chlorides, phosphates, hydrogen phosphates, dihydrogen phosphates, hydroxides, or salts of organic acids, such as, for example, acetates, fumarates, maleates, citrates, etc. The further pharmacologically active constituents can also be present in the form of hydrates or solvates. Phosphorus is preferably added in the form of phosphates or hydrogen phosphates.

Because ascorbic acid is oxidised with atmospheric oxygen at neutral pH to give dehydroascorbic acid, preparations in the form of conventional solutions that are exposed to the air are not very suitable to not at all suitable according to the invention.

However, preparations that are stable over a longer period, such as tablets (chewing tablets, film-coated tablets, effervescent tablets), effervescent granules, powder mixtures, capsules, sachets, and also kits in which the iron(III) complex and optionally further constituents are present in solution, for example in single-portion vials or bottles, and the redox-active substance(s), in particular ascorbic acid, preferably in powder or granule form, is/are added immediately before consumption, are very suitable. Water inter alia is used as solvent for the last-mentioned solutions, but conventional syrup bases or juices are also suitable. Particular preference is given according to the invention to single-dose containers, i.e. vessels, preferably made of glass, whose lid is designed as a container for the redox-active substances, which are then introduced and mixed with the contents shortly before consumption by pushing the bottom of the lid into the vessel. Such single-dose containers are known and available commercially.

It is further provided according to the invention to consume the iron(III) complex compound simultaneously with or close in terms of time to the redox-active substance(s), in particular ascorbic acid, the redox-active substance(s) preferably being consumed in the form of a solution, particularly preferably in the form of fruit juice, in particular orange juice.

Close in terms of time here means that the two components are administered at an interval of not more than 2 hours, preferably not more than 30 minutes.

There are preferably consumed from 40 mg to 120 mg, more preferably from 60 mg to 100 mg, of iron(III) (calculated as iron(III), not as iron(III) complex) in the form of a tablet, capsule, drop, juice, dragée or other oral galenic preparation, with 100 ml of orange juice, corresponding to an ascorbic acid content of about 150 mg. Particular preference is given to tablets comprising 60 mg or 100 mg of iron(III). Further pharmacologically active constituents as mentioned above can optionally be present either in the preparation of the iron(III) complex or in the solution of the redox-active substance(s) or in both.

The iron(III) hydroxide complex compounds and the redox-active substance(s), and optionally further constituents, can be brought into the suitable pharmaceutical form with conventional pharmaceutical carriers or auxiliary substances. Conventional binders or lubricants, diluents, disintegrators, fillers, etc. can be used for this purpose. Tablets can be coated with conventional film-forming agents. Flavourings, taste-imparting substances and colourings can also be added, if desired.

The iron(III) hydroxide complex compounds used in accordance with the invention are administered orally. The daily dose is, for example, from 10 to 500 mg of iron(III)/day of administration. Patients with iron deficiency or iron deficiency anaemia consume, for example, 100 mg of iron(III) from 2 to 3 times daily, and pregnant women consume 60 mg of iron(III) from 1 to 2 times daily (in each case calculated as iron(III), not as complex).

The daily dose of redox-active compound, in particular ascorbic acid, is, for example, from 50 to 300 mg daily, preferably approximately 150 mg, which roughly corresponds to one glass of orange juice.

The preparation can be administered over a period of several months, without hesitation, until the iron status improves, as reflected by the patient's haemoglobin value, transferrin saturation and ferritin value, or until the desired improvement in brain power or immune response is achieved or the symptoms of restless legs syndrome improve.

The preparation according to the invention can be taken by children, young people and adults.

The use according to the invention takes place in particular by improving the iron, haemoglobin, ferritin and transferrin values. An improvement in the short-term memory test (STM), in the long-term memory test (LTM), in the Raven's progressive matrices test, in the Welscher adult intelligence scale (WAIS) and/or in the emotional coefficient (Baron EQ-i, YV test; youth version) or an improvement in the neutrophil level, the antibody level and/or the lymphocyte function.

The mode of action of the invention is explained and demonstrated by the following examples.

### EXAMPLES

### Example 1

Film-coated tablets each comprising the following constituents were prepared in the conventional manner:

| | | |
|---|---|---|
| β-carotene | 7.2 | mg |
| vitamin B1 (as thiamine nitrate) | 2.0 | mg |
| vitamin B2 | 1.8 | mg |
| vitamin B6 (as pyridoxine hydrochloride) | 2.7 | mg |
| vitamin B12 | 0.0026 | mg |
| ascorbic acid | 95 | mg |
| vitamin D3 | 10 | μg |
| vitamin E | 12 | mg |
| biotin | 0.1 | mg |
| calcium pantothenate | 7.6 | mg |
| nicotinamide | 20 | mg |
| folic acid | 0.8 | mg |
| copper sulfate, anhydrous | 5 | mg |
| manganese chloride tetrahydrate | 11 | mg |
| zinc sulfate monohydrate | 52 | mg |
| calcium hydrogen phosphate, anhydrous | 439 | mg |
| magnesium oxide | 166 | mg |
| iron(III)-polymaltose complex | 226 | mg (60 mg Fe(III)) |
| Croscarmellose sodium | 41 | mg |
| colloidal anhydrous silicon oxide | 7 | mg |
| magnesium stearate | 6 | mg |
| microcrystalline cellulose | 116 | mg |
| Opadry 85F27316 (film-forming agent) | 50 | mg |

### Example 2

Film-coated tablets each comprising the following constituents were prepared in the conventional manner:

| | |
|---|---|
| iron(III)-polymaltose complex | 226 mg (60 mg Fe(III)) |
| ascorbic acid | 95 mg |
| Croscarmellose sodium | 41 mg |
| colloidal anhydrous silicon oxide | 7 mg |
| magnesium stearate | 6 mg |
| microcrystalline cellulose | 116 mg |
| Opadry 85F27316 (film-forming agent) | 50 mg |

### Example 3

The following measurements were obtained when iron(III)-polymaltose complex with ascorbic acid (ascorbic acid) was investigated at pH 3.0, 5.5 and 8.0:

| | | | Formation of |
|---|---|---|---|
| | Reaction time | Formation of Fe²⁺ | dehydroascorbic |
| pH | [h] | ions [%] | acid [%] |
| 3.0 | 2 | 2 | 2 |
| | 4 | 7 | 7 |
| 5.5 | 2 | 0 | 0 |
| 8.0 | 2 | 5 | 9 |
| | 4 | 5 | 13 |

The buffered solutions (buffers as described above, see Geisser, P., Arneim.-Forsch./Drug Res. 1990, 40 (II), 7, 754-760) were mixed in a molar ratio of Fe(III):ascorbic acid of 1:1, the concentration of Fe(III) and ascorbic acid in the mixture being 5×10⁻⁵ mol/l in each case, and investigated using a conventional UV-VIS spectrophotometer. The operation was carried out with the strict exclusion of oxygen.

The table clearly shows that iron(III)-polymaltose complex with ascorbic acid reacts only slowly within a period of 4 hours to dehydroascorbic acid and Fe(II) at pH values of from 3 to 8.

### Example 4

### Clinical Study

The effect of freshly squeezed orange juice (enhancer) and tea (inhibitor) on the absorption of labelled ⁵⁹Fe in erythrocytes after the oral administration of iron(III)-polymaltose complex to subjects with and without iron deficiency was studied.

### Method:

This is a single-centre cross-over study. Each test subject took part in two periods during which a single dose of 100 mg of iron was administered as ⁵⁹Fe-labelled iron(III)-polymaltose complex (labelled Maltofer® (Vifor (International) AG, Switzerland)). During one period, the test subjects fasted overnight prior to administration of the preparation; during the other, they received specific food (group A and group B) before the medicament was administered. As an alternative, the medication was administered in the saturated state with an iron-absorption enhancer (orange juice) or an iron-absorption inhibitor (black tea) (group C and group D). A total of 32 subjects took part in the study. They were both healthy subjects and subjects with iron deficiency. In detail, the groups were divided as follows:

Group A: Subjects with iron deficiency, who received the test medicament, successively, in each case in the state after standardised food consumption or after fasting overnight.

Group B: Normal test subjects, who received the test medicament, successively, in each case after standardised food consumption or after fasting overnight.

Group C: Subjects with iron deficiency, who received the test medicament, successively, in the state after standardised food consumption together with orange juice or with black tea.

Group D: Normal subjects, who received the test medicament, successively, in the state after standardised food consumption together with orange juice or with black tea.

In groups A and B, the test medicament was administered together with 100 ml of tap water, either on an empty stomach (i.e. after fasting overnight) or after a standardised breakfast.

In groups C and D, the test medicament was administered after a standardised breakfast with 100 ml of freshly squeezed orange juice (corresponding to an ascorbic acid content, determined by conventional methods, of 150 mg) or with 100 ml of black tea.

In all groups there were administered for the test, and thereby consumed together, in each case 2 ml of ⁵⁹Fe-labelled Maltofer® drops, corresponding to 100 mg of iron, in each case in 100 ml of water, orange juice or black tea (Earl Grey, 1 tea bag to 100 ml of water, allow to brew for 4 minutes). The cup used was immediately rinsed with 100 ml of water and this water was also drunk.

When the test medicament was administered in the state after food consumption, the standardised breakfast was served 30 minutes prior to administration and had to be finished within 30 minutes.

All test subjects additionally received standardised lunches, afternoon snacks and evening meals about 4, 6 and 9 hours, respectively, after administration of the test medicament.

The test persons fulfilled the following requirements:
haemoglobin <130 g/l (iron deficiency) or ≧130 g/l (normal)
transferrin saturation <16% or ferritin <30 μg/l (iron deficiency), transferrin saturation 16% or ferritin ≧30 μg/l (normal)
no further cause for anaemia (thalassaemia, malignant tumours, chronic infections, etc.)

### Test Product

100 mg of iron were administered orally as 2 ml of ⁵⁹Fe-labelled iron(III)-polymaltose complex solution (Maltofer® drops, Vifor (International) AG, Switzerland) in a concentration of 50 mg of elemental iron/ml. It is a macromolecular complex (molecular weight 53,200 daltons). Labelling of the test medicament Maltofer® drops obtained from the manufacturer was carried out at the GIN Laboratory, Uppsala University, Sweden according to our own preparation specification corresponding to GMP and GLP. Two single doses were administered in each case, separated by an excretion period of at least 21 days. The radioactivity administered in the two periods was:

period 1: 1 MBq ⁵⁹Fe
period 2: 2 MBq ⁵⁹Fe

### Pharmacokinetics and Efficiency

The primary pharmacokinetics and efficiency variable was the incorporation of ⁵⁹Fe in erythrocytes. The secondary end-point was the ⁵⁹Fe activity in plasma. Samples for determination of ⁵⁹Fe in plasma and erythrocytes were taken 96 hours after administration of the medicament and on days 7, 14 and 21 following administration of the medicament. The samples were placed in EDTA tubes and centrifuged within a period of 60 minutes, samples of plasma and erythrocytes were stored cooled until analysed.

The following parameters were measured:
haematology: blood haemoglobin, blood haematocrit, blood leukocyte count, blood RBC, blood WBC, erythrocyte MCV, MCH, MCHC, blood platelets, serum TIBC, serum Fe, serum transferrin saturation, serum ferritin
clinical chemistry: serum cyanocobalamin (B₁₂), serum folate, RBC folate.

A plateau in the erythrocyte uptake curve was expected at about 20 days/3 weeks after administration of the test medicament. The blood volume was determined from the height and weight of the test subjects according to Nadler et al. (Nadler, S. B., Surgery, 1962, 224-232). In order to calculate the total amount of ⁵⁹Fe circulating in the blood, the measured blood radioactivity concentration was multiplied by the blood volume. This value was divided by the amount of ⁵⁹Fe administered in order to calculate the primary efficiency end-point, i.e. the percentage of the administered dose that had been incorporated.

The uptake values after 3 weeks were then used for the statistical evaluation. 2 MBq were administered in the second treatment period, and a residual noise from the administration of 1 MBq during the first period of study could be expected. The 3-week uptake value after administration of 2 MBq in the second period was therefore corrected by subtracting the 3-week uptake value after administration of 1 MBq in the first period.

A comparison of the absorption of ⁵⁹Fe between anaemic and normal test subjects was also carried out, and an assessment was made of the ⁵⁹Fe activity in the plasma after oral administration of iron(II)-polymaltose complex by descriptive plasma-time-activity profiles.

Evaluation of the data was carried out by conventional statistical methods.

### Summary of the Results

Expressed as the relative incorporation of iron in erythrocyes, both test subjects with iron deficiency and those without iron deficiency benefited from the simultaneous administration of the orange juice enhancer with Maltofer® drops.

A comparison of the iron uptake in erythrocytes between anaemic and normal test subjects was carried out by the Student's test in the 5% level (one-sided).

The results of the erythrocyte uptake after treatment with Maltofer® drops after fasting and after food consumption are as follows:

TABLE 2

| | | Iron deficiency | | No iron deficiency | |
|---|---|---|---|---|---|
| | | After | After food | After | After food |
| Parameter | | fasting | consumption | fasting | consumption |
| ⁵⁹Fe uptake | N | 8 | 8 | 8 | 8 |
| in | Mean | 1.615 | 1.941 | 1.411 | 0.924 |
| erythrocytes | Geom. | 0.766 | 1.165 | 1.208 | 0.866 |
| (%) | mean | | | | |
| | SD | 1.539 | 1.184 | 0.842 | 0.299 |
| | Median | 1.5 | 1.23 | 1.10 | 0.98 |
| | Min. | 0.06 | 0.24 | 0.51 | 0.33 |
| | Max. | 4.25 | 4.83 | 2.85 | 1.39 |

The results of the erythrocyte uptake after administration of Maltofer® drops with orange juice (enhancer) or black tea (inhibitor) are as follows:

TABLE 3

| | | Iron deficiency | | No iron deficiency | |
|---|---|---|---|---|---|
| Parameter | | Inhibitor | Enhancer | Inhibitor | Enhancer |
| ⁵⁹Fe uptake | N | 8 | 8 | 8 | 8 |
| in | Mean | 4.105 | 6.588 | 1.381 | 1.864 |
| erythrocytes | Geom. | 2.510 | 4.530 | 0.928 | 1.279 |
| (%) | mean | | | | |
| | SD | 4.342 | 7.159 | 1.126 | 1.590 |
| | Median | 2.58 | 4.37 | 1.17 | 1.23 |
| | Min. | 0.33 | 1.26 | 0.25 | 0.33 |
| | Max. | 13.83 | 23.52 | 3.06 | 4.38 |

The point estimation and the 90% confidence interval of the ratio of the geometric means for the relative incorporation of iron in erythrocytes between the state after fasting and after food consumption and between inhibitor and enhancer (PP Set) are as follows:

TABLE 4

| | | | 90% confidence | |
|---|---|---|---|---|
| State of | | Point | interval | |
| subject | Ratio | estimation | Upper | Lower |
| Iron | Fasting/food | 0.66 | 0.34 | 1.28 |
| deficiency | | | | |
| Iron | Inhibitor/enhancer | 0.55 | 0.36 | 0.86 |
| deficiency | | | | |
| No iron | Fasting/food | 1.39 | 0.97 | 1.99 |
| deficiency | | | | |
| No iron | Inhibitor/enhancer | 0.73 | 0.41 | 1.28 |
| deficiency | | | | |

The point estimation, the p value and the 90% confidence interval of the ratio of the geometric mean for the relative incorporation of iron in erythrocytes between anaemic and normal subjects after fasting, after food consumption, with an inhibitor and with an enhancer are as follows:

TABLE 5

| | | | p value | 90% confidence | |
|---|---|---|---|---|---|
| State of | | Point | (one- | interval | |
| subject | Ratio | estimation | sided) | lower | upper |
| Fasting | ID/ND¹ | 0.63 | 0.2324 | 0.22 | 1.81 |
| Food | ID/ND¹ | 1.35 | 0.2551 | 0.63 | 2.88 |
| With | ID/ND¹ | 2.70 | 0.0440 | 1.04 | 7.03 |
| inhibitor | | | | | |
| With | ID/ND¹ | 3.54 | 0.0082 | 1.56 | 8.02 |
| enhancer | | | | | |

¹ID = iron deficiency; ND = no iron deficiency

The means, given in Tables 2 and 3, of the iron uptake in erythrocytes in subjects with iron deficiency and those without iron deficiency, after fasting, after food consumption and with orange juice are summarised in the table below.

TABLE 6

| Iron deficiency | | | | | | | |
|---|---|---|---|---|---|---|---|
| Orange | | | | No iron deficiency | | | |
| juice | Tea | Fasting | Food | Orange juice | Tea | Fasting | Food |
| 6.58 | 4.105 | 1.615 | 1.941 | 1.84 | 1.381 | 1.411 | 0.924 |

The results show that iron absorption (relative incorporation of iron in erythrocytes) in subjects with iron deficiency was improved when Maltofer® was administered with food or orange juice, the effect of orange juice on the iron absorption being markedly greater than that of food. An effect of the inhibitor compared with food is not discernible. Normal test subjects also benefited from orange juice compared with treatment with Maltofer® together with an inhibitor, although the effect is less than in subjects with iron deficiency. In normal test subjects, the iron absorption after fasting was greater than on administration together with food, the reverse of the situation in subjects with iron deficiency.

It has further been confirmed that in subjects with iron deficiency, the iron absorption on administration of Maltofer® together with food, an enhancer or inhibitor was greater than in normal subjects. Greater absorption was observed in normal subjects when Maltofer® was administered after fasting.

No severe side-effects of Maltofer® drops were found during the study. The most frequent side-effects were headaches, diarrhoea and stomach ache in mild or moderate form; no severe side-effects were observed.

1-18. (canceled)

19. A method for treating iron deficiency states consisting of administering, to a patient having an iron deficiency state, a preparation consisting of
(i) at least one iron(III) complex compound,
(ii) ascorbic acid,
(iii) optionally one or more further pharmacologically active constituents selected from the group consisting of vitamins, with the exception of ascorbic acid, trace elements, cofactors, minerals and nutrients, and
(iv) optionally conventional pharmaceutical carriers or auxiliary substances selected from conventional binders, lubricants, diluents, disintegrators, fillers,
wherein the iron(III) complex compounds have a redox potential at pH 7 of from −324 mV to −750 mV relative to a normal hydrogen electrode, and
wherein the preparation is suitable for oral administration.

20. The method of claim 19, wherein the preparation is in a form selected from the group consisting of tablets, granules, capsules, effervescent tablets, a powder mixture, effervescent granules, a sachet, and combinations thereof.

21. The method of claim 19, wherein the at least one iron (III) polymaltose complex compound and the ascorbic acid are present in a weight ratio of 1:0.4 to 1:1.8.

22. The method of claim 21, wherein the patient is selected from the group consisting of patients with chronic inflammatory bowel disease, patients with Crohn's disease patients with Colitis ulcerosa, and patients who are pregnant women.
